(19) United States
(12) Patent Application Publication
(10) Pub. No.: US 2022/0064332 A1
(43) Pub. Date: Mar. 3, 2022

(54) METHODS AND COMPOSITIONS FOR TREATING CANCERS BY IMMUNO-MODULATION USING ANTIBODIES AGAINST CATHESPIN-D

(71) Applicants: INSERM (INSTITUT NATIONAL DE LA SANTÉ ET DE LA RECHERCHE MÉDICALE), Paris (FR); INSTITUT RÉGIONAL DU CANCER DE MONTPELLIER, Montpellier (FR); UNIVERSITÉ DE MONTPELLIER, Montpellier (FR)

(72) Inventors: Emmanuelle LIAUDET-COOPMAN, Montpellier (FR); Hanane MANSOURI, Grenoble (FR); Nathalie BONNEFOY, Montpellier (FR); Henri-Alexandre MICHAUD, Montpellier (FR); Pascal ROGER, Montpellier (FR); Lindsay ALXARAZ CACCHIA, Montpellier (FR); Séverine GUIU, Montpellier (FR); Valérie LAURENT-MATHA, Montpellier (FR)

(21) Appl. No.: 17/414,707

(22) PCT Filed: Dec. 18, 2019
(86) PCT No.: PCT/EP2019/085833

§ 371 Date: Jun. 16, 2021

(30) Foreign Application Priority Data
Dec. 19, 2018 (EP) 18306735.4

(51) Int. Cl. C07K 16/40; A61K 45/06; A61P 35/00

(52) U.S. Cl. CPC C07K 16/40; A61P 35/00; A61K 45/06

(57) ABSTRACT

Inventors have generated two human anti-cath-D scFv fragments cloned in the human IgG1λ format (F1 and E2) that efficiently bind to human and mouse cath-D, even at the acidic pH of the TNBC microenvironment. F1 and E2 accumulated in TNBC MDAMB-231 tumor xenografts, inhibited tumor growth and improved mice survival without apparent toxicity. Using this xenograft model, they found that the Fc function of F1 was essential for maximal tumor inhibition. Inventors have shown that the anti-cath-D antibody F1 treatment prevented the recruitment of tumor-associated macrophages and myeloid-derived suppressor cells within the tumor, a specific effect associated with a less immunosuppressive tumor microenvironment. Moreover F1 inhibited tumor growth of TNBC patient-derived xenografts (PDXs). This preclinical proof-of-concept study validates the feasibility and efficacy of an immunomodulatory antibody-based strategy against cath-D to treat patients with TNBC. Accordingly, the present invention relates to an anti-cath-D antibody which inhibits the tumor recruitment of immunosuppressive tumor-associated macrophages M2 and myeloid-derived suppressor cells for use in the treatment of cancer.

### FIELD OF THE INVENTION

The present invention relates to cancer field. More particularly, the invention relates to use of anti-cathepsin-D antibodies in the treatment of cancers, particularly of triple negative breast cancer.

### BACKGROUND OF THE INVENTION

Breast cancer (BC) is one of the leading causes of death in women in developed countries. Triple negative breast cancer (TNBC), defined by the absence of estrogen receptor (ER), progesterone receptor (PR) and human epidermal growth factor receptor 2 (HER-2) overexpression and/or amplification, accounts for 15-20% of all BC cases (1). Chemotherapy is the primary systemic treatment, but resistance to this treatment is common (1). Hence, tumor-specific molecular targets and/or alternative therapeutic strategies for TNBC are urgently needed. With the discovery of antigens specifically expressed in TNBC cells and the developing technology of monoclonal antibodies, immunotherapy is emerging as a novel promising option for TNBC (2).

Human cathepsin D (cath-D) is a ubiquitous, lysosomal, aspartic endoproteinase that is proteolytically active at low pH. Cath-D is overproduced and abundantly secreted by human epithelial BC cells (3) with expression levels in BC correlating with poor prognosis (4, 5). Cath-D affects both cancer and stromal cells in the breast tumor microenvironment by increasing BC cell proliferation (3, 6, 7), fibroblast outgrowth (8, 9), tumor angiogenesis (10, 11), tumor growth and metastasis (6). Human cath-D is synthesized as a 52-kDa precursor that is converted into an active 48-kDa single-chain intermediate in the endosomes, and then into a fully active mature form, composed of a 34-kDa heavy chain and a 14-kDa light chain, in the lysosomes. Its catalytic site includes two critical aspartic residues, residue 33 on the 14-kDa chain and residue 231 on the 34-kDa chain.

The over-production of cath-D by BC cells leads to hypersecretion of the 52-kDa pro-cath-D into the extracellular environment (3). Purified 52-kDa pro-cath-D auto-activates in acidic conditions giving rise to a catalytically active 51-kDa pseudo-cath-D that retains the 18 residues (27-44) of the pro-segment (12). Extracellular cath-D displays oncogenic activities by proteolysis at acidic pH and via nonproteolytic mechanisms through protein-protein interaction (13-15). Extracellular cath-D can modify the local extracellular matrix by cleaving chemokines (16, 17), growth factors, collagens, fibronectin, proteoglycans, protease inhibitors (e.g., cystatin C (18), PAI 1 (19)), or by activating enzyme precursors (e.g., cathepsins B and L) (13). It can also promote BC cell proliferation by binding to an unknown receptor via the residues 27-44 of its pro-peptide (20). It can trigger breast fibroblast outgrowth upon binding to the LRP1 receptor (9, 21), and induce endothelial cell proliferation and migration via the ERK and AKT signaling pathways (11). Therefore, extracellular cath-D could represent a novel molecular target in BC. Cath-D deficiency in humans is associated with neuronal ceroid lipofuscinosis, one amongst the most common pediatric neurodegenerative lysosomal storage diseases, indicating its non-redundant essential role in protein catabolism and cellular homeostasis maintenance (22). Consequently concomitant inhibition of intracellular and extracellular cath-D with cell-permeable chemical drugs (23) could be toxic. Therefore, identifying specific molecular targets and/or alternative therapeutic strategies for TNBC is urgently needed.

### SUMMARY OF THE INVENTION

The present invention relates to a human anti-cathepsin-D antibody which inhibits the tumor recruitment of immunosuppressive tumor-associated macrophages M2 and myeloid-derived suppressor cells for use in the treatment of cancer. In particular, the invention is defined by the claims.

### DETAILED DESCRIPTION OF THE INVENTION

Inventors have generated two human anti-cath-D scFv fragments cloned in the human IgG1λ format (F1 and E2) that efficiently bind to human and mouse cath-D, even at the acidic pH of the TNBC microenvironment. Anti-cath-D F1 and E2 antibodies accumulated in TNBC MDA-MB-231 tumor xenografts, inhibited tumor growth and improved mice survival without apparent toxicity. Using this xenograft model, they found that the Fc function of F1 was essential for maximal tumor inhibition.

For the first time, inventors have shown that the F1 antibody prevented the recruitment of tumor-associated macrophages M2 (TAMs) and myeloid-derived suppressor cells (MDSCs) within the tumor, a specific effect associated with a less immunosuppressive tumor microenvironment. The F1 antibody inhibited tumor growth of TNBC patient-derived xenografts (PDXs).

This preclinical proof of-concept study validates the feasibility and efficacy of an anti-cath-D immunomodulatory antibody based strategy to treat patients with TNBC.

Accordingly, in a first aspect, the invention relates to a human anti-cathepsin-D antibody which inhibits the tumor recruitment of immunosuppressive tumor-associated macrophages M2 and myeloid-derived suppressor cells for use in the treatment of cancer.

In a particular embodiment, the invention relates to a method for treating cancer in a subject in need thereof comprising a step of administering said subject with a therapeutically effective amount of an antibody anti-cath-D antibody or a fragment thereof.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “immunosuppressive tumor-associated macrophages M2” also known as M2 macrophages or Tumor-associated macrophages type M2 (TAM-M2) is a type of blood-borne phagocytes, derived from circulating monocytes or resident tissue macrophages. Their complex roles in carcinogenesis generally lead to disease progression in many cancers, which share some similar pathological mechanisms. There are two different subpopulations of activated macrophages within tumor microenvironment. The first type, known as classically activated macrophages (M1 macrophages or TAM-M1), are activated by lipopolysaccharides (LPS) or by double signals from interferon (IFN)-γ and tumor necrosis factor-α (TNF-α). This first type of macrophage are able to kill microorganisms and tumor cells.

The second type of macrophages is known as alternatively activated macrophages (M2 macrophages or TAM-2). Exposure to IL-4, IL-13, vitamin D3, glucocorticoids or transforming growth factor-β (TGF-β) decreases macrophage antigen-presenting capability and up-regulates the expression of macrophage mannose receptors (MMR, also known as CD206), scavenger receptors (SR-A, also known as CD204), dectin-1 and DC-SIGN.9 M2-polarized macrophages exhibit an IL-12^{low}, IL-23^{low}, IL-10^{high} phenotype. This second type of macrophage plays an important role in stroma formation, tissue repair, tumor growth, angiogenesis and immunosuppression.

In BC, TAMs are the most abundant inflammatory cells and are typically M2-polarized with suppressive capacity (1) that stems from their enzymatic activities and production of anti-inflammatory cytokines, such as TGFβ (Fuxe et al., Semin Cancer Biol, 2012, 22:455-461). High TAM levels have been associated with poorer BC outcomes (Zhao et al., Oncotarget, 2017, 8:30576-86. Therefore, several strategies are currently under investigation, such as the suppression of TAM recruitment, their depletion, or the switch from the pro-tumor M2 to the anti-tumor M1 phenotype in patients with TNBC (Georgoudaki et al., Cell Reports, 2016, 15:2000-11). Our findings showing reduced macrophage infiltration and decreased M2-like macrophages in response to F1 treatment are in line with the ongoing therapeutic strategies.

As used herein, the term “myeloid-derived suppressor cells” (MDSC) refers to a group of immune cells from the myeloid lineage. They have immature state and ability to potently suppress T cell responses. They regulate immune responses and tissue repair in healthy individuals and the population rapidly expands during inflammation, infection and cancer. MDSCs are immature myeloid cells that promote the immunosuppressive tumor microenvironment through multiple mechanisms, including expression of immunosuppressive cytokines, such as TGFβ (Gabrilovich et al., Nature Rev Immunol, 2009, 9:162-74).

In the context of the invention, the anti-cath-D F1 antibody as described below is able to inhibit the recruitment of immunosuppressive immune cells such as TAM and MDSC.

As used herein, the term “cath-D” has its general meaning in the art and refers to lysosomal aspartic protease cathepsin-D. Cath-D is synthesized as the 52 kDa, catalytically inactive, precursor called pro-cath-D. It is present in endosomes as an active 48 kDa single-chain intermediate that is subsequently converted in the lysosomes into the fully active mature protease, composed of a 34 kDa heavy and a 14 kDa light chains. In cancer, the 52-kDa pro-form is oversecreted. The naturally occurring pro-cath-D protein has an amino acid sequence shown in Genbank, Accession number NP 001900.

As used herein, the term “anti-cath-D antibody” refers to an antibody directed against cath-D.

As used herein, the terms “antibody” or “immunoglobulin” have the same meaning, and will be used equally in the invention. The term “antibody” as used herein refers to immunoglobulin molecules and immunologically active portions of immunoglobulin molecules, i.e., molecules that contain an antigen binding site that immune-specifically binds to an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG (encompassing distinct subclasses such as IgG1, IgG2, IgG3 and IgG4), IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

As used herein, the term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

As used herein, the term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

As used herein, the term “Fab′” refers to an antibody fragment having a molecular weight of about 50,000 and antigen binding activity, which is obtained by cutting a disulfide bond of the hinge region of the F(ab′)2.

As used herein, the term “single chain Fv” (“scFv”) polypeptide is a covalently linked VH::VL heterodimer which is usually expressed from a gene fusion including VH and VL encoding genes linked by a peptide-encoding linker.

As used herein, the term “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

As used herein, the term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

In a particular embodiment, the antibody is a monoclonal human antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

As used herein, the terms “neutralizing antibody” refers to an antibody that blocks or reduces at least one activity of a polypeptide comprising the epitope to which the antibody specifically binds. A neutralizing antibody reduces Cathepsin D biological activity in in cellulo and/or in vivo tests. Typically, an anti-Cath-D neutralizing antibody fragment blocks Cath-D binding to LRP1 (which can be assessed by GST pull-down assays) and/or also inhibits catalytic activity of mature Cath-D (which can be assessed by a catalytic activity assay based on the cleavage reaction by Cath-D of a fluorogenic substrate such as M2295 (fluorogenic peptide substrate for pseudo-Cath-D) or M0938 (fluorogenic peptide substrate for mature Cath-D) as described below.

The term “LRP1” has its general meaning in the art (Strickland and Ranganathan, 2003; Lillis et al., 2005) and refers to LDL receptor-related protein 1. LRP1 is composed of a 515 kDa extracellular α chain and an 85 kDa β chain generated by proteolytic cleavage from a 600 kDa precursor polypeptide in a trans-Golgi compartment. Actually, LRP1 α chain and LRP1 β chain are issued from a sole transcript. By way of example, the human full length of unprocessed precursor LRP1 corresponds to SwissProt accession number Q07954.

By “purified” and “isolated” it is meant, when referring to an antibody according to the invention, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present.

In the context of the invention, the amino acid residues of the antibody of the invention are numbered according to the IMGT numbering system. The IMGT unique numbering has been defined to compare the variable domains whatever the antigen receptor, the chain type, or the species (Lefranc M.-P., “Unique database numbering system for immunogenetic analysis” Immunology Today, 18, 509 (1997); Lefranc M.-P., “The IMGT unique numbering for Immunoglobulins, T cell receptors and Ig-like domains” The Immunologist, 7, 132-136 (1999); Lefranc, M.-P., Pommié, C., Ruiz, M., Giudicelli, V., Foulquier, E., Truong, L., Thouvenin-Contet, V. and Lefranc, G., “IMGT unique numbering for immunoglobulin and T cell receptor variable domains and Ig superfamily V-like domains” Dev. Comp. Immunol., 27, 55-77 (2003).). In the IMGT unique numbering, the conserved amino acids always have the same position, for instance cysteine 23, tryptophan 41, hydrophobic amino acid 89, cysteine 104, phenylalanine or tryptophan 118. The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc. (http://www.imgt.org/IMGTScientific Chart/Nomenclature/IMGT-FRCDRdefinition.html)

In the context of the invention, inventors have isolated by antibody phage display two fully human anti-Cath-D single-chain variable antibody fragment (scFv), selected on human cellular mature (34+14-kDa) Cath-D, referred as F1 and E2.

The inventors have cloned and characterized the variable domain of the light and heavy chains of said scFv F1, and thus determined the complementary determining regions (CDRs) domain of said antibody as described in Table 1:

TABLE 1
sequences of ScFv F1 antibody

| ScFv F1 | |
|---|---|
| Domains | Sequence SEQ ID NO: |
| VH | SEQ ID NO: 1 |
| | EVQLVESGGSLVKPGGSLRLSCAASGFTSNNYMNWVRQAP |
| | GKGLEWISYISGSSRYISYADFVKGRFTISRDNATNSLYL |
| | QMNSLRAEDTAVYYCVRSSNSGGMDVWGRGTLVTVSS |
| VH-CDR1 | SEQ ID NO: 2 |
| | GFTFSNNY |
| VH-CDR2 | SEQ ID NO: 3 |
| | ISGSSRYI |
| VH-CDR3 | SEQ ID NO: 4 |
| | VRSSNSGGMDV |
| VL | SEQ ID NO: 5 |
| | QSVLTQPASVSGSPGQSITISCAGTSSDVGGYYGVSWYQQ |
| | HPGKAPKLMIYYDSYRPSGVSNRFSGSKSGNTASLTISGL |
| | QAEDEADYYCSSYTSNSTRVFGGGTKLAVL |
| VL-CDR1 | SEQ ID NO: 6 |
| | SSDVGGYYG |
| VL-CDR2 | SEQ ID NO: 7 |
| | GDS |
| VL-CDR3 | SEQ ID NO: 8 |
| | SSYTSNSTRV |

The antibody for use according to the invention, wherein said antibody comprising: a) a heavy chain CDR1 comprising the amino acid sequence of SEQ ID NO: 2, b) a heavy chain CDR2 comprising the amino acid sequence of SEQ ID NO: 3, c) a heavy chain CDR3 comprising the amino acid sequence of SEQ ID NO: 4; d) a light chain CDR1 comprising the amino acid sequence of SEQ ID NO: 6; e) a light chain CDR2 comprising the amino acid sequence of SEQ ID NO: 7; and f) a light chain CDR3 comprising the amino acid sequence of SEQ ID NO: 8.

The antibody for use according to the invention, wherein said antibody comprising: a) a heavy chain wherein the variable domain has a sequence set forth as SEQ ID NO:1 and b) a light chain wherein the variable domain has a sequence set forth as SEQ ID NO:5.

The inventors have also cloned and characterized the variable domain of the light and heavy chains of said scFv E2, and thus determined the complementary determining regions (CDRs) domain of said antibody as described in Table 2:

TABLE 2
sequences of ScFv E2 antibody

| ScFv E2 | |
|---|---|
| Domains | Sequence SEQ ID NO: |
| VH | SEQ ID NO: 9 |
| | EVQLVESGGSLVKPGGSLRLSCAASGFTFSNSYMNWVRQAP |
| | GKGLEWISYISGSSRYSYADFVKGRFTISRDNATNSLYLQM |
| | NSLRAEDTAVYYCVRSSNSYFGGGMDVWGRGTLVTVSS |
| VH-CDR1 | SEQ ID NO: 10 |
| | GFTFSNSY |
| VH-CDR2 | SEQ ID NO: 11 |
| | ISGSSRYI |
| VH-CDR3 | SEQ ID NO: 12 |
| | VRSSNSYFGGGMDV |
| VL | SEQ ID NO: 13 |
| | QSVLTQPASVSGSPGQSITISCAGTSSDVGGSYGVSWYQQH |
| | PGKAPKLMIYGDSYRPSGVSNRFSGSKSGNTASLTISGLQA |
| | EDEADYYCSSYTNYSTRVFGGGTKLAVL |
| VL-CDR1 | SEQ ID NO: 14 |
| | SSDVGGSYG |
| VL-CDR2 | SEQ ID NO: 15 |
| | GDS |
| VL-CDR3 | SEQ ID NO: 16 |
| | SSYTNYSTRV |

The antibody for use according to the invention, wherein said antibody comprising: a) a heavy chain CDR1 comprising the amino acid sequence of SEQ ID NO: 10, b) a heavy chain CDR2 comprising the amino acid sequence of SEQ ID NO: 11, c) a heavy chain CDR3 comprising the amino acid sequence of SEQ ID NO: 12; d) a light chain CDR1 comprising the amino acid sequence of SEQ ID NO: 14; e) a light chain CDR2 comprising the amino acid sequence of SEQ ID NO: 15; and f) a light chain CDR3 comprising the amino acid sequence of SEQ ID NO: 16.

The antibody for use according to the invention, wherein said antibody comprising: a) a heavy chain wherein the variable domain has a sequence set forth as SEQ ID NO:9 and b) a light chain wherein the variable domain has a sequence set forth as SEQ ID NO:13

In some embodiments, the glycosylation of the antibody of the invention is modified. Glycosylation can be altered to, for example, increase the affinity of the antibody for the antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al.

It should be also noted that the antibodies F1 and E2 cross-react with murin cath-D, which is of interest for preclinical evaluation and toxicological studies.

It should be further noted that the F1 and E2 antibodies (e.g. with the IgG1 isotype) specifically bind to cath-D, and do not bind with others aspartic proteases (e.g. cathepsin E, pepsinogen A and pepsinogen C).

In a particular embodiment, the anti-cath-D antibody of the invention is able to induce cytotoxicity, also known as the antibody-dependent cell-mediated cytotoxicity (ADCC).

ADCC is a mechanism of cell-mediated immune defense whereby an effector cell of the immune system actively lyses a target cell, whose membrane-surface antigens have been bound by specific antibodies. Typically, in the context of the invention, the anti-cath-D F1 antibody as described above is able to activate NK cells (up-regulation of cytolytic enzymes-granzyme B and perforin, and the anti-tumor cytokine TNFα), suggesting the occurrence of ADCC in vivo.

In particular embodiment, the antibody comprising: a) a heavy chain CDR1 comprising the amino acid sequence of SEQ ID NO: 2, b) a heavy chain CDR2 comprising the amino acid sequence of SEQ ID NO: 3, c) a heavy chain CDR3 comprising the amino acid sequence of SEQ ID NO: 4; d) a light chain CDR1 comprising the amino acid sequence of SEQ ID NO: 6; e) a light chain CDR2 comprising the amino acid sequence of SEQ ID NO: 7; and f) a light chain CDR3 comprising the amino acid sequence of SEQ ID NO: 8 is able to induce cytotoxicity.

In particular embodiment, the antibody comprising: a) a heavy chain wherein the variable domain has a sequence set forth as SEQ ID NO: 1 and b) a light chain wherein the variable domain has a sequence set forth as SEQ ID NO: 5 is able to induce cytotoxicity.

In particular embodiment, the antibody comprising: a) a heavy chain CDR1 comprising the amino acid sequence of SEQ ID NO: 10, b) a heavy chain CDR2 comprising the amino acid sequence of SEQ ID NO: 11, c) a heavy chain CDR3 comprising the amino acid sequence of SEQ ID NO: 12; d) a light chain CDR1 comprising the amino acid sequence of SEQ ID NO: 14; e) a light chain CDR2 comprising the amino acid sequence of SEQ ID NO: 15; and f) a light chain CDR3 comprising the amino acid sequence of SEQ ID NO: 16 is able to induce cytotoxicity.

In particular, the antibody comprising: a) a heavy chain wherein the variable domain has a sequence set forth as SEQ ID NO:9 and b) a light chain wherein the variable domain has a sequence set forth as SEQ ID NO:13 is able to induce cytotoxicity.

Accordingly, the anti-cath-D antibody of the invention is able to activate NK cells.

In particular embodiment, the antibody comprising: a) a heavy chain CDR1 comprising the amino acid sequence of SEQ ID NO: 2, b) a heavy chain CDR2 comprising the amino acid sequence of SEQ ID NO: 3, c) a heavy chain CDR3 comprising the amino acid sequence of SEQ ID NO: 4; d) a light chain CDR1 comprising the amino acid sequence of SEQ ID NO: 6; e) a light chain CDR2 comprising the amino acid sequence of SEQ ID NO: 7; and f) a light chain CDR3 comprising the amino acid sequence of SEQ ID NO: 8 is able to activate NK cells.

In particular embodiment, the antibody comprising: a) a heavy chain wherein the variable domain has a sequence set forth as SEQ ID NO: 1 and b) a light chain wherein the variable domain has a sequence set forth as SEQ ID NO: 5 is able to activate NK cells.

In particular embodiment, the antibody comprising: a) a heavy chain CDR1 comprising the amino acid sequence of SEQ ID NO: 10, b) a heavy chain CDR2 comprising the amino acid sequence of SEQ ID NO: 11, c) a heavy chain CDR3 comprising the amino acid sequence of SEQ ID NO: 12; d) a light chain CDR1 comprising the amino acid sequence of SEQ ID NO: 14; e) a light chain CDR2 comprising the amino acid sequence of SEQ ID NO: 15; and f) a light chain CDR3 comprising the amino acid sequence of SEQ ID NO: 16 is able to activate NK cells.

In particular, the antibody comprising: a) a heavy chain wherein the variable domain has a sequence set forth as SEQ ID NO:9 and b) a light chain wherein the variable domain has a sequence set forth as SEQ ID NO:13 is able to activate NK cells.

In a particular embodiment, the present invention relates to a nucleic acid sequence encoding a heavy chain or light chain of the antibody for use according to the invention.

In another embodiment, the present invention relates to a vector comprising a nucleic acid according to the invention.

In another embodiment, the present invention relates to a host cell comprising a nucleic acid according to the invention or a vector according to the invention.

In a particular embodiment, the antibody anti-Cath-D is conjugated to the drugs. Said antibody is called as antibody drug conjugate (ADC). In a particular embodiment, such antibody is combined with the potency of chemotherapeutic agents. The technology associated with the development of monoclonal antibodies to tumor associated target molecules, the use of more effective cytotoxic agents, and the design of chemical linkers to covalently bind these components, has progressed rapidly in recent years (Ducry L, et a/. Bioconjugate Chemistry, 21:5-13, 2010). An “anti-Cath-D antibody-drug conjugate” as used herein refers to an anti-Cath-D antibody according to the invention conjugated to a therapeutic agent. Such anti-Cath-D antibody-drug conjugates produce clinically beneficial effects on Cath-D-expressing cells when administered to a patient, such as, for example, a patient with a Cath-D-expressing cancer, typically when administered alone but also in combination with other therapeutic agents.

In typical embodiments, an anti-Cath-D antibody is conjugated to a cytotoxic agent, such that the resulting antibody-drug conjugate exerts a cytotoxic or cytostatic effect on a Cath-D-expressing cell (e.g., a Cath-D-expressing cancer cell) when taken up or internalized by the cell. Particularly suitable moieties for conjugation to antibodies are chemotherapeutic agents, prodrug converting enzymes, radioactive isotopes or compounds, or toxins. For example, an anti-Cath-D antibody can be conjugated to a cytotoxic agent such as a chemotherapeutic agent or a toxin (e.g., a cytostatic or cytocidal agent such as, for example, abrin, ricin A, pseudomonas exotoxin, or diphtheria toxin).

Useful classes of cytotoxic agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and-carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, camptothecin, carboplatin, carmustine (BSNU), CC-1065 (Li et al., Cancer Res. 42:999-1004, 1982), chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, etopside phosphate (VP-16), 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide (VM-26), 6-thioguanine, thioTEPA, topotecan, vinblastine, vincristine, and vinorelbine.

Particularly suitable cytotoxic agents include, for example, dolastatins (e.g., auristatin E, AFP, MMAF, MMAE), DNA minor groove binders (e.g., enediynes and lexitropsins), duocarmycins, taxanes (e.g., paclitaxel and docetaxel), puromycins, vinca alkaloids, CC-1065, SN-38 (7-ethyl-10-hydroxy-camptothein), topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone. In certain embodiments, a cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be linked to an anti-Cath-D antibody.

In specific variations, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP (dimethylvaline-valine-dolaisoleuine-dolaproine-phenylalanine-p-phenylenediamine), MMAF (dovaline-valine-dolaisoleunine-dolaproine-phenylalanine), and MAE (monomethyl auristatin E). The synthesis and structure of auristatin E and its derivatives are described in U.S. Patent Application Publication No. 20030083263; International Patent Publication Nos. WO 2002/088172 and WO 2004/010957; and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In other variations, the cytotoxic agent is a DNA minor groove binding agent. (See, e.g., U.S. Pat. No. 6,130,237.) For example, in certain embodiments, the minor groove binding agent is a CBI compound. In other embodiments, the minor groove binding agent is an enediyne (e.g., calicheamicin).

In certain embodiments, an antibody-drug conjugate comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, an anti-Cath-D antibody is conjugated to a pro-drug converting enzyme. The pro-drug converting enzyme can be recombinantly fused to the antibody or chemically conjugated thereto using known methods. Exemplary pro-drug converting enzymes are carboxypeptidase G2, β-glucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Arnon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.)

In a particular embodiment, the antibody or a fragment thereof according to the invention for use as a drug.

In a particular embodiment, the antibody or a fragment thereof according to any of according to the invention for use in the treatment of cancer and neuronal diseases. Diseases associated with cath-D overexpression are particularly cancers. The antibodies of the invention may be used alone or in combination with any suitable agent.

In another embodiment, the antibody or a fragment thereof for use in the treatment of hyperproliferative diseases. More particularly, the hyperproliferative diseases are associated with cath-D overexpression.

As used herein, the term “abnormal cell growth” and “hyperproliferative disorders or diseases” are used interchangeably in this application and refers to cell growth that is independent of normal regulatory mechanisms (e.g., loss of contact inhibition). In the context of the invention the hyperproliferative diseases refers to diseases having an overexpression of cathepsin-D. Typically, hyperproliferative diseases are selected but not limited to, cancer (e.g. breast cancer, renal cancer etc), skin disorders (e.g. psoriasis, wound healing), inflammatory diseases (e.g. inflammatory bowel disease).

In a particular embodiment, the hyperproliferative disease is cancer. As used herein, the term “cancer” refers to a group of diseases involving abnormal cell growth with the potential to invade or spread to other parts of the body. The cancer that may treated by methods and compositions of the invention include, but are not limited to cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyo sarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment, the cancer includes, but is not limited to breast cancer, melanoma, ovarian cancer, lung cancer, liver cancer, pancreatic cancer, endometrial cancer, head and neck cancer, bladder cancer, malignant glioma, prostate cancer, colon adenocarcinoma or gastric cancer.

In a particular embodiment, the breast cancer is an estrogen-receptor positive (ER+) hormone-resistant breast cancer or a triple-negative (ER− and PR−, HER2-non amplified) breast cancer (TNBC).

In a further embodiment, the antibody or a fragment thereof according to the invention for use in the treatment of neurological, neuropathic or psychiatric disorders. Typically, the antibody or a fragment thereof according to the invention for use in the treatment of schizophrenia, cerebral ischemia, stroke, neuropathic pain, spinal cord injury, Alzheimer's disease, Parkinson's disease, and/or multiple sclerosis.

As used herein, the term “subject” refers to any mammals, such as a rodent, a feline, a canine, and a primate. Particularly, in the present invention, the subject is a human afflicted with or susceptible to be afflicted with a disease wherein Cath-D is overexpressed. In another embodiment, the subject is a human afflicted with or susceptible to be afflicted with a cancer. In another embodiment, the subject is a human afflicted with or susceptible to be afflicted with TNBC.

As used herein the terms “administering” or “administration” refer to the act of injecting or otherwise physically delivering a substance as it exists outside the body (e.g., an anti-cath-D antibody) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of an anti-cath-D antibody for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Immune checkpoints are the regulators of the immune system. They are crucial for self-tolerance, which prevents the immune system from attacking cells indiscriminately. Immune checkpoints are targets for cancer immunotherapy due to their potential for use in multiple types of cancers. Typically, by using immune checkpoint inhibitors, the anti-tumoral response is reactivated by reactivation of cytotoxic T-lymphocytes. The anti-cath-D antibody as described above can be combined with an immune checkpoint inhibitor to inhibit the recruitment of immunosuppressive tumor-associated macrophages M2 and myeloid-derived suppressor cells.

Accordingly, in a second aspect, the invention relates to a combined preparation comprising the antibody for use according to the invention and an immune checkpoint inhibitor.

In a particular embodiment, the combined preparation according to the invention for use in the treatment of cancer.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint molecules include CD27, CD28, CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-éthyl]amino}-1,2,5-oxadiazole-3 carboximidamide:

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

The antibody for use according to the invention and the immune checkpoint inhibitor as described above are administered to the subject in need thereof simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

The antibody for use according to the invention alone and/or combined with an immune check point inhibitor as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: Anti-cath-D antibody-based therapy prevents macrophage recruitment within MDA-MB-231 tumor xenografts. (A) Tumor growth. When MDA-MB-231 tumor xenografts reached a volume of 50 mm3, nude mice were treated with F1 (n=9), E2 (n=9), or rituximab (CTRL; n=9) (15 mg/kg) for 28 days (day 16-44). At day 44, all mice were sacrificed. ***, P=0.001 for F1; **, P=0.002 for E2 (mixed-effects ML regression test). (B) Mean tumor volume at day 44. n=9 for CTRL; n=9 for F1; n=9 for E2. ***, P=0.0001 for F1; **, P=0.0012 for E2 (Student's t test); mean±SEM. (C) Representative images of F4/80 immunostaining in MDA-MB-231 tumor cell xenografts from CTRL- (rituximab), F1- and E2-treated mice. Scale bars, 100 (D) Linear regression analysis of F4/80+ macrophages and tumor volumes. R2=0.1553; *, P=0.0464, n=27.

FIG. 2: Anti-cath-D antibody-based therapy prevents M2-like macrophage and MDSC recruitment, and triggers anti-tumor response via NK cell activation in MDA-MB-231 xenografts. (A) Tumor growth. Nude mice bearing MDA-MB-231 tumors of 50 mm3 were treated with F1 (n=9), F1Fc (n=8), or rituximab (CTRL; n=9) (15 mg/kg) for 35 days. At day 54, mice were sacrificed. *, P<0.001 for F1 versus CTRL; P=0.077 for F1Fc versus CTRL; P=0.069 for F1 versus F1Fc (mixed effects ML regression test). (B) Mean tumor volumes at day 54. Mean±SEM; *, P=0.011 for F1 versus CTRL; P=0.231 for F1Fc versus CTRL, P=0.189 for F1 versus F1Fc (Student's t-test). (C) TAM recruitment. The percentage of F4/80+ CD11b+ TAMs was quantified by FACS and expressed relative to all CD45+ immune cells (n=9 for CTRL; n=9 for F1; n=8 for F1Fc); *, P=0.044 for F1 versus CTRL; P=0.3 for F1Fc versus CTRL (Student's t-test). (D) Linear regression analysis of TAM and tumor volumes. R2=0.5425; ***, P<0.0001; n=26. (E) Quantification of CD206 mRNA expression. Total RNA was extracted from MDA-MB-231 tumor xenografts at the end of treatment, and CD206 expression analyzed by RT-qPCR and shown relative to F4/80 (n=9 for CTRL; n=9 for F1; n=8 for F1Fc); P=0.05 for F1 versus CTRL; P=0.04 for F1Fc versus CTRL (Student's t-test). (F) MDSC recruitment. The percentage of Gr1+ CD11b+ MDSCs was quantified by FACS analysis and expressed relative to all CD45+ cells (n=9 for CTRL; n=9 for F1; n=8 for F1Fc); **, P=0.008 for F1 versus CTRL; P=0.079 for F1Fc versus CTRL (Student's t-test). (G) Linear regression analysis of MDSC and tumor volumes. R2=0.23315; *, P=0.0125; n=26. (H) Quantification of TGFβ mRNA expression. Total RNA was extracted from MDA-MB-231 tumor cell xenografts at the end of treatment and TGFβ expression analyzed by RT-qPCR. Data are relative to RPS9 expression (n=9 for CTRL; n=9 for F1; n=8 for F1Fc); **, P=0.009 for F1 versus CTRL; P=0.1 for F1Fc versus CTRL (Student's t-test). (I) NK recruitment. The percentage of CD49b+ CD11b+ NK cells was quantified by FACS and expressed relative to all CD45+ cells (mean±SEM; n=9 for rituximab (CTRL); n=9 for F1; n=8 for F1Fc); P=0.7 for F1 versus CTRL; P=0.8 for F1Fc versus CTRL; P=0.8 for F1 versus F1Fc (Student's t-test). (J) Quantification of IL-15 mRNA expression. Total RNA was extracted from MDA-MB-231 tumor cell xenografts at the end of treatment and IL-15 analyzed by RT-qPCR. Data are the mean±SEM expression level relative to RPS9 expression (n=9 for rituximab (CTRL); n=9 for F1; n=8 for F1Fc. **, P=0.0013 for F1 versus CTRL; P=0.365 for F1Fc versus CTRL; *, P=0.0127 for F1 versus F1Fc (Student's t-test). (K) Linear regression analysis of IL-15 mRNA level and tumor volumes. R2=0.3693; **, P=0.0013; n=26. (L) Quantification of granzyme B mRNA expression as in (B). ***, P=0.0002 for F1 versus CTRL; **, P=0.0011 for F1Fc versus CTRL; **, P=0.0076 for F1 versus F1Fc (Student's t-test). (M) Quantification of perforin mRNA expression as in (B). *, P=0.033 for F1 versus CTRL; *, P=0.0294 for F1Fc versus CTRL; P=0.386 for F1 versus F1Fc (Student's t-test). (N) Quantification of IFNγ mRNA expression as in (B). ***, P<0.0001 for F1 versus CTRL; P=0.0513 for F1Fc versus CTRL; **, P=0.0078 for F1 versus F1Fc (Student's t-test).

FIG. 3: Therapeutic effects of F1 in mice engrafted with PDX B1995 or PDX 3977. Mice were engrafted with PDX B1995 (left panel) or PDX B3977 (right panel) and when tumor volumes reached 150 mm3, mice were treated with F1 (15 mg/kg) or NaCl (CTRL) three times per week. Mice were sacrificed when tumor volume reached 2000 mm3 and the corresponding tumor growth curves were stopped. Tumor volume (in mm3) is shown as the mean±SEM; For B1995 PDX: n=7 for CTRL; n=7 for F1. ***, P<0.001 for F1. For B3977 PDX: n=10 for CTRL; n=10 for F1. *, P=0.022 for F1.

### EXAMPLE

Material & Methods

Reagents

The anti-human cath-D antibody against 52-, 48-, and 34-kDa forms was from Transduction Laboratories (#610801BD). The anti-human cath-D antibody (ab75811) against 14-kDa form was from Abcam. The anti-human cath-D antibody against 4-kDa pro-domain was kindly provided by Prof M. Fusek (Oklahoma Medical Research Foundation). The anti-human cath-D antibodies M1G8, D7E3 and M2E8 were previously described (9, 24). The anti-human cath-D antibody (clone C-5) and CD11c (HL3) were from Santa Cruz Biotechnology. The anti-human Fc antibody conjugated to HRP (A0170) was from Sigma Aldrich. The anti-human CD20 chimeric IgG1 antibody (rituximab) was from Roche, and the anti-mouse F4/80 antibody (clone BM8, MF48000) from Invitrogen. Matrigel (10 mg/ml) was purchased from Corning. The fluorescent-conjugated antibodies against CD45 (30-F11), F4/80 (BM8), CD11b (M1/70), and Gr1 (RB6-8C5) were from Thermo Fisher Scientific, and against CD49b (DX5), and MHC-II (M5/114.15.2) were from Abcam. Recombinant human pro-cath-D was purchased from R&D Systems.

Cell Lines, ELISA, Immunoprecipitation and Western Blotting

The MDA-MB-231 cell line was previously described (6). Cells were cultured in DMEM with 10% fetal calf serum (FCS, GibcoBRL). To produce conditioned medium, cells were grown to 90% confluence in DMEM medium with 10% FCS, and conditioned medium was centrifuged at 800×g for 10 min. For sandwich ELISA, 96-well plates were coated with M2E8 antibody in PBS (500 ng/well) at 4° C. overnight. After blocking non-specific sites with PBST/1% BSA, conditioned medium was added at 4° C. for 2 h. After washes in PBST, serial dilutions of F1 or E2 were added at 4° C. for 2 h and interaction revealed with an anti-human Fc antibody conjugated to HRP (1/2000; 355 ng/well). Cath-D was quantified in TNBC cytosols by sandwich ELISA, as described above, after coating with the D7E3 antibody in PBS (200 ng/well) and with the M1G8 antibody conjugated to HRP (1/80), and using recombinant pro-cath-D (1.25-15 ng/ml) for reference (6). TNBC cytosols were previously prepared and frozen (25). GST-cath-D fusion proteins were produced in the E. coli B strain BL21 as described (9). The resulting proteins were separated on 12% SDS-PAGE and analyzed by immunoblotting.

In Vivo Studies

MDA-MB-231 cells (2×106; mixed 1:1 with Matrigel) were injected subcutaneously in 6-week-old female athymic mice (Foxn1nu, ENVIGO). When tumors reached a volume of about 50 mm3, tumor bearing mice were randomized and treated with F1 (15 mg/kg), E2 (15 mg/kg), rituximab (15 mg/kg), or NaCl by intraperitoneal injection 3 times per week. Tumors were measured using a caliper and volume was calculated using the formula V=(tumor length×tumor width×tumor depth)/2, until the tumor volume reached 2000 mm3. For PDX models, approximately 5×5×5 mm of B1995 and B3977 tumor fragments were transplanted in the inter-scapular fat pads of in 6-week-old female Foxn1nu mice. When tumor volume reached a volume of about 150 mm3, mice were randomized in two treatments groups: F1 (15 mg/kg) or saline solution by intraperitoneal injection 3 times per week. Tumor volumes were measured as described above.

SPECT/CT Imaging

To generate 177Lu-labeled antibodies, F1 and E2 were conjugated with p-SCN-benzyl-DOTA. The immunoreactivity of the DOTA-conjugated antibodies (5 and 7 DOTA/IgG for F1 and E2, respectively) was verified by ELISA. DOTA-conjugated F1 and E2 were then labeled with 177Lu (Perkin Elmer) at 200 MBq/mg. Radiochemical purity was >97% and radionuclide purity >99.94%. For SPECT-CT imaging, 5 mice were xenografted with MDA-MB-231 cells. When tumors reached a volume of about 150 mm3, mice received an intraperitoneal injection of 7 MBq of 177Lu-F1 or 177Lu-E2 (3 mice for F1 and 2 for E2). At 24, 48, and 72 h post-injection, whole-body SPECT/CT images were acquired using a four-headed NanoSPECT imager (Bioscan Inc., Washington D.C.). Reconstructed data from SPECT and CT images were visualized and co-registered using Invivoscope®.

Immunohistochemistry

For cath-D immunostaining, TNBC TMA and PDX primary tumor sections were incubated with anticath-D mouse antibody (clone C-5) at 0.4 μg/ml for 20 min after heat-induced antigen retrieval using the PTLink pre-treatment (Dako) and the High pH Buffer (Dako) and endogenous peroxidase quenching with Flex Peroxidase Block (Dako). After two rinses in EnVision™ Flex Wash buffer (Dako), sections were incubated with a HRP-labeled polymer coupled to secondary anti-mouse antibody (Flex® system, Dako) for 20 min, followed by 3,3′-diaminobenzidine as chromogen. Sections were counterstained with Flex Hematoxylin (Dako) and mounted after dehydration. Sections were analyzed independently by two experienced pathologists, both blinded to the tumor characteristics and patient outcomes at the time of scoring. Tumor and normal epithelial breast cells with peripheral membrane labeling were scored as positive for single-labeled cells. Extracellular granulations observed in the stroma were considered as extracellular cath-D staining. Extracellular cath-D was defined as negative in the presence of 0 to 5% of stromal extracellular signal, and positive for values above 6%. For IHC of MDA-MB-231 xenografts, tumor samples were collected and fixed in 10% neutral buffered formalin for 24 h, dehydrated, and embedded in paraffin. For F4/80 immunostaining, xenograft sections (4-μm thick), sections were incubated with an anti-F4/80 antibody for 30 min, followed by a rabbit anti-rat antibody (Thermo Scientific, 31218) before the Envision® system (Dako). Diaminobenzidine (Dako) as described above. F4/80 staining images were digitalized with the NanoZoomer slide scanner (Hamamatsu) and analysed with the Aperio Imagescope software.

Homology Modeling and Docking

Homology models were built using Modeller (27). The heavy and light chain (VH and VL) were modeled separately, using as template the closest homolog with the same CDR length. VH and VL models were then reassembled based on the relative orientation in the template used for VH modeling. Docking of each molecular model on cath-D was made using PRIOR (28). Figures were prepared using the PyMOL Molecular Graphics System (Version 2.0 Schrödinger, LLC).

Gene Expression Data Analysis

Recurrence-free survival with a 10-year follow-up was calculated using the on-line Kmplot tool accessed on Oct. 2 2017 with the 200766_at Affymetrix probe ((28), www.http://kmplot.com). Analysis was restricted to the 255 patients with TNBC present in the database at this date and with the best cut-off option. Differences were evaluated with the Log-rank test.

Quantitative RT-PCR

Reverse transcription of total RNA was performed at 37° C. using the Moloney murine leukemia virus reverse transcriptase (Invitrogen, Carlsbad, Calif.) and random hexanucleotide primers (Promega, Madison, Wis.). Real-time quantitative PCR analyses were performed on a Light Cycler 480 SYBR Green I master and a Light Cycler 480 apparatus (both from Roche Diagnostics, Indianapolis, Ind.). The PCR product integrity was verified by melting curve analysis. Quantification data were normalized to the amplification data for the reference gene encoding ribosomal protein S9 (RPS9). The sequences of the primers for IL-15, GZMB, PRF1, IFNγ, CD206, F4/80, TGFβ, and RPS9 are in Table S1 (data not shown).

Isolation of Tumor-Infiltrating Cells and FACs Analysis

Tumors were digested with a mixture of collagenase IV (1 mg/ml) (Sigma) and DNase I (200 U/ml) (Sigma) in Hank's Balanced Salt Solution (HBSS) containing 2% FCS at 37° C. for three incubations of 15 min/each. The mixtures were then mechanically separated using the Gentle MACs procedure. After digestion, tumor suspensions were passed through a 70 μm nylon cell strainer, centrifuged and resuspended in FACS buffer (PBS pH 7.2, 1% FBS, 2 mM EDTA and 0.02% sodium azide). Cells were blocked with FACS buffer containing 1% (v/v) of Fc Block (Miltenyi) and, stained with fluorescent conjugated antibodies against the following cell surface markers: CD45, CD49b, F4/80, CD11c, CD11b, Gr1 and MHC-II. MDSCs were defined as CD45posCD49bnegCD11cnegCD11bposGr1posMHC-IIneg cells. Dendritic cells were defined as CD45posCD49bnegCD11cposMHC-IIneg cells. Macrophages were defined as CD45posCD11bposF4/80pos cells within the gate excluding MDSCs and dendritic cells. Sorted cells were then washed in FACS buffer, and fixed with 1% PFA in PBS. Samples were analyzed by flow cytometry using a Beckman and Coulter Cytoflex flow cytometer. Tumor cells were defined as CD45− negative events in a scatter gate that included small and large cells. Events were analyzed with FlowJo 10.4.

Statistical Analysis.

A linear mixed regression model was used to determine the relationship between tumor growth and number of days after xenograft. The variables included in the fixed part of the model were the number of days post-graft and the treatment group; their interaction was also evaluated. Random intercepts and random slopes were included to take into account the time effect. The model coefficients were estimated by maximum likelihood. A survival analysis was conducted, and the event considered was a tumor volume of 2000 mm3. Survival rates were estimated using the Kaplan-Meier method and survival curves were compared with the Log-rank test. Statistical analysis was conducted with the STATA 13.0 software. The Student's t test was used to evaluate difference. Statistical significance was set at the 0.05 level.

Results

Cath-D within the Tumor Microenvironment is Eligible for Antibody-Mediated Targeted Therapy in TNBC Patients

First, we investigated the clinical significance of the expression of CTSD (the gene encoding cath-D) in a cohort of 255 patients with TNBC using an online survival analysis (29). High CTSD mRNA level was significantly associated with shorter recurrence-free survival (HR=1.65 [1.08-2.53]; p=0.019) (data not shown), suggesting that cath-D overexpression could be used as a predictive marker of poor TNBC prognosis.

Then, to assess whether cath-D in TNBC was an accessible molecular target for anti-cath-D antibodies, we re-analyzed cath-D status in previously published datasets used for biotin-based affinity isolation and proteomic analysis of accessible protein biomarkers in human BC tissues (30) and (data not shown). We found that extracellular and/or membrane-associated cath-D could be detected only in the TNBC tumor sample and not in the adjacent normal breast tissue (data not shown). We validated these proteomic data by anti-cath-D immunohistochemistry (IHC) analysis of a Tissue Micro-Array (TMA) that included 123 TNBC (data not shown). We detected extracellular cath-D in the microenvironment of 98% of TNBC samples (data not shown) and at the cancer cell surface of 85.7% of samples (data not shown). Conversely, extracellular and membrane-associated cath-D expression was very weak in normal breast tissues (data not shown). Together with the previously published data, our results show that cath-D is a tumor cell-associated extracellular biomarker and strongly suggest that it could be a good candidate for antibody-based therapy in TNBC.

Selection of Novel Anti-Human Cath-D scFv Fragments by Phage Display

For future potential clinical application especially in patients with TNBC, we decided to engineer a collection of novel, fully human, anti-cath-D antibodies. For this purpose, we probed the phage antibody expression library Husc I (31, 32) with recombinant human 34+14-kDa cath-D, and isolated polyclonal human antibodies in scFv format showing specific binding to immobilized cath-D by ELISA. After enrichment by four rounds of bio-panning (data not shown), we selected five monoclonal antibodies based on their binding to recombinant human 52-kDa pro-cath-D and 34+14-kDa mature cath-D (data not shown). We purified these five his-tagged scFv fragments by affinity chromatography (data not shown), and determined by ELISA that the purified antibodies still bound to secreted human 52-kDa pro-cath-D and cellular cath-D (data not shown) from MDA-MB-231 cells (cell line derived from an invasive ductal cell carcinoma that represents one of the most common TNBC models). These scFv fragments also recognized mouse cellular cath-D (81.1% of identity with human cath-D) from MEF cells (data not shown). We then used the three scFv antibodies (F1, E2 and E12 scFv) with the highest binding to human and mouse cath-D to produce fully human IgG1λ (F1, E2, E12).

Generation of Anti-Cath-D Human Antibodies

Sandwich ELISA using pro-cath-D secreted from MDA-MB-231 cells showed that F1 (data not shown) and E2 (data not shown) retained good binding capacities (EC50=0.2 nM and 1.2 nM, respectively). Conversely, E12 in the IgG1 format lost its binding activity (data not shown). Moreover, F1 and E2 binding to pro-cath-D was comparable at pH values from 7.5 to 5.5 (data not shown), suggesting that they are active also in the highly acidic tumor microenvironment. We also confirmed F1 and E2 good selectivity towards pro-cath-D compared with other aspartic enzymes, such as pro-cathepsin E, pepsinogen A and pepsinogen C (data not shown). We next characterized the cath-D epitope recognized by F1 and E2. Molecular docking performed on the three-dimensional structure of mature cath-D (PDB ID 1LYA) (33) showed that F1 and E2 scFv interacted mainly with the 34-kDa cath-D chain (in red) (data not shown). Moreover, the third complementary determining region of the heavy chain (CDRH3) of both F1 and E2 scFv, which is crucial for antibody specificity, protruded into the proteinase active site (data not shown). By competitive ELISA, we confirmed that F1 and E2 epitopes overlapped (data not shown). Finally, using GSTcath-D fusion fragments, we showed that both F1 and E2 immunoprecipitated the 52-, 48- and 34-kDa forms of GST-cath-D, but not the 4-kDa GST-cath-D pro-fragment and the 14-kDa light chain GSTcath-D (data not shown). These results indicated that, the cath-D epitopes of F1 and E2 are located mainly on the 34-kDa part of the protein (data not shown).

Anti-Cath-D Human Antibodies Localize and Accumulate in MDA-MB-231 Tumor Xenografts

We then assessed F1 and E2 localization by SPECT/CT and their bio-distribution in nude mice xenografted subcutaneously with MDA-MB-231 cells. When tumor cell xenografts reached about 150 mm3, mice received one single intraperitoneal injection of antibodies labeled with lutetium 177 (177Lu-F1 and 177Lu-E2), a radionuclide emitting gamma particles that can be used for imaging and biodistribution purposes. Whole-body SPECT/CT images acquired 24, 48 and 72 h post-injection showed that 177Lu-F1 and 177Lu-E2 accumulated in the MDA-MB-231 tumor xenografts (data not shown). The bio-distribution profiles confirmed that 177Lu-F1 and 177Lu-E2 gradually accumulated in the tumors from 24 h and up to 96 h (data not shown). The percentage (mean±SD) of injected activity/g tissue detected in tumors (% IA/g) at 72 h was 8.2%±4.3% for F1 and 8.1%±2.2% for E2 (data not shown). Moreover, at 72 h, 177Lu-F1 and 177Lu-E2 were present also in blood (6.4%±2.1% and 11.3%±6.1%, respectively), and liver (10.5%±5.2% and 10.7%±4.7%, respectively). However, their concentration in blood and liver decreased rapidly due to physiological elimination. These results indicate that F1 and E2 localize and accumulate in TNBC MDA-MB-231 xenografts.

The Anti-Cath-D F1 and E2 Antibodies Inhibit TNBC MDA-MB-231 Tumor Growth and Improve Survival

We used athymic Foxn1nu nude mice xenografted subcutaneously with MDA-MB-231 cells to study the anti-tumor properties of the anti-cath-D antibodies F1 and E2. When MDA-MB-231 tumors reached 50 mm3, we treated mice with F1, E2 (15 mg/kg), or saline solution (control) by intraperitoneal injection 3 times per week for 32 days (day 23-55 post-graft), and sacrificed them when tumor volume reached 2000 mm3. Treatment with F1 or E2 significantly delayed tumor growth compared with control (data not shown). At day 55, tumor volume was reduced by 58% in the F1 (P=0.0005) and by 49% (P=0.0026) in the E2 group compared with control (data not shown). Moreover, the overall survival rate, reflected by a tumor volume superior to 2000 mm3, was significantly longer in mice treated with F1 or E2 than in controls, with a median survival of 72 and 64 days for the F1 and E2 groups respectively, compared with 57 days for control animals (data not shown). These results show that anti-cath-D human antibodies as monotherapy delay very efficiently tumor growth in nude mice xenografted with MDA-MD-231 cells.

Anti-Cath-D Antibody-Based Therapy Prevents Macrophage Recruitment within MDA-MB-231 Tumor Xenografts

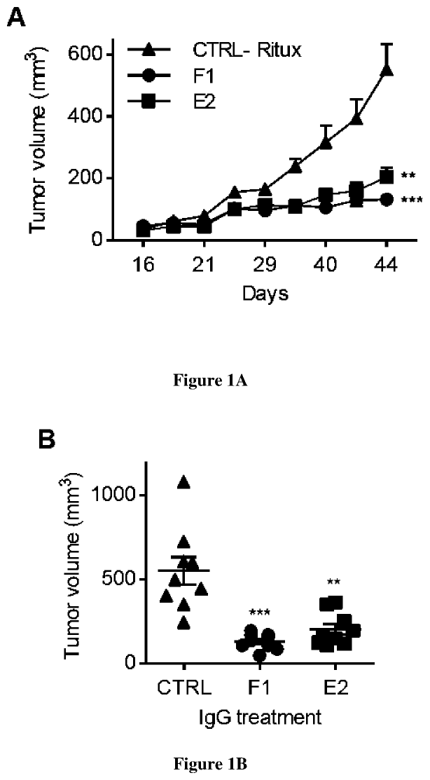

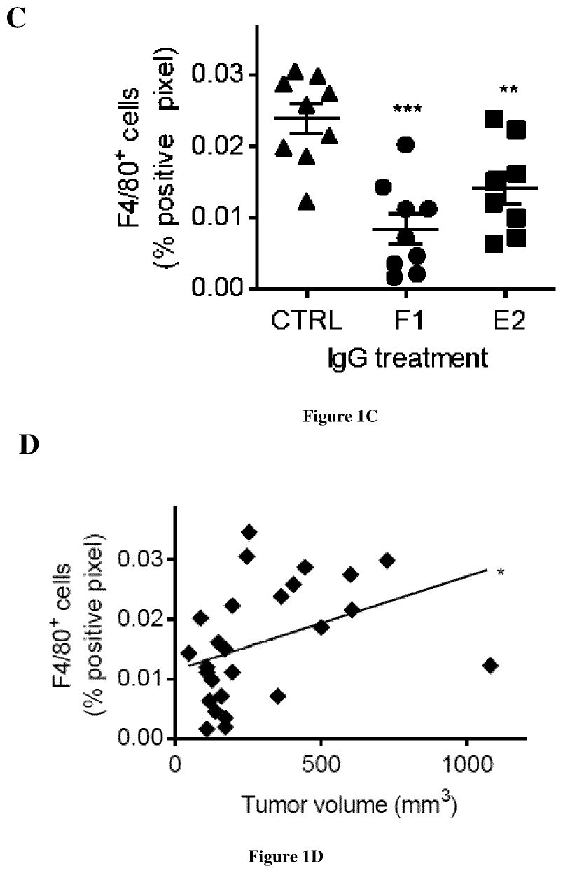

To further investigate the in vivo mechanisms underlying the antitumor effect of F1 and E2, we treated nude mice xenografted with MDA-MB-231 cells with F1, E2 or the anti-human CD20 IgG1 rituximab, as negative isotype control (same schedule as before), and then sacrificed all mice at the treatment end. F1 and E2 led to a significant inhibition of tumor growth compared with rituximab (P=0.001 for F1, P=0.002 for E2) (FIG. 1A). At the end of the experiment (day 44), tumor volume was reduced by 76% in the F1 group (P=0.0001) and by 63% (P=0.0012) in the E2 group, compared with the rituximab group (FIG. 1B). Moreover, although F1 and E2 cross-react with mouse cath-D, mice treated with the anti-cath-D antibodies gained weight and displayed normal activities (data not shown), suggesting minimal off-target effects for these human antibodies.

Then, we investigated the effect of F1 and E2 monotherapy on tumor cell proliferation, apoptosis, and angiogenesis by IHC. Ki67, a marker of proliferating cells (data not shown), activated caspase 3, a marker of apoptosis (data not shown), and the angiogenesis marker CD31 (data not shown) were similarly expressed in tumors from the three groups of mice. As antibody-based immunotherapy is often associated with immune modulation of the tumor microenvironment (34), we assessed the impact of anti-cath-D antibodies on tumor-infiltrating immune cells, particularly on myeloid cells that are present in Foxn1nu nude mice. Staining with the antimacrophage F4/80 antibody revealed that macrophage infiltration in the tumor core was reduced by 64.8% in the F1 and by 41% in the E2 group, compared with the rituximab group (data not shown). Moreover, the percentage of F4/80+ cells was positively associated with tumor volume by linear regression analysis (FIG. 1D; P=0.0464). Our findings show that anti-cath-D antibody treatment inhibit macrophage infiltration in MDA-MD-231 tumor xenografts, suggesting that this antibody-based therapy may impact the tumor immune microenvironment.

The Anti-Cath-D Antibody F1 Prevents M2-Like Macrophages and MDSC Recruitment, Leading to a Less Immunosuppressive Tumor Microenvironment in MDA-MB-231 Xenografts

As the immunomodulatory effect of antibody-based therapy could depend on Fc-mediated mechanisms (35), we engineered an aglycosylated Fc-silent version of the F1 antibody (F1Fc) in which the mutation N297A prevents binding to FcγRs (36).

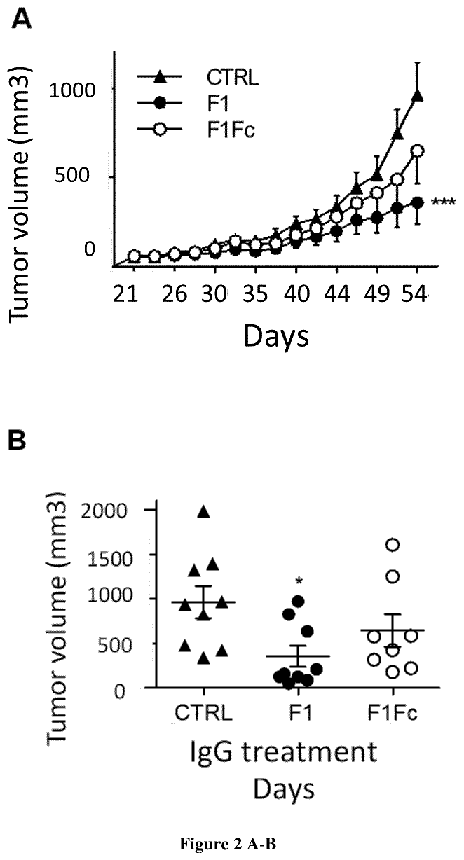

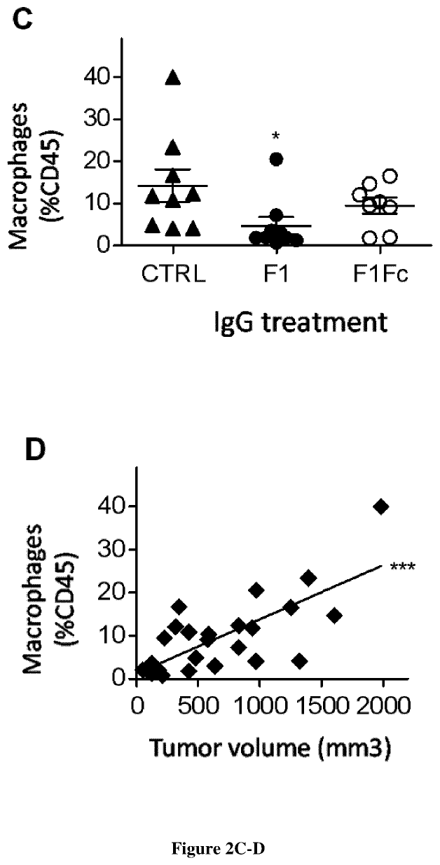

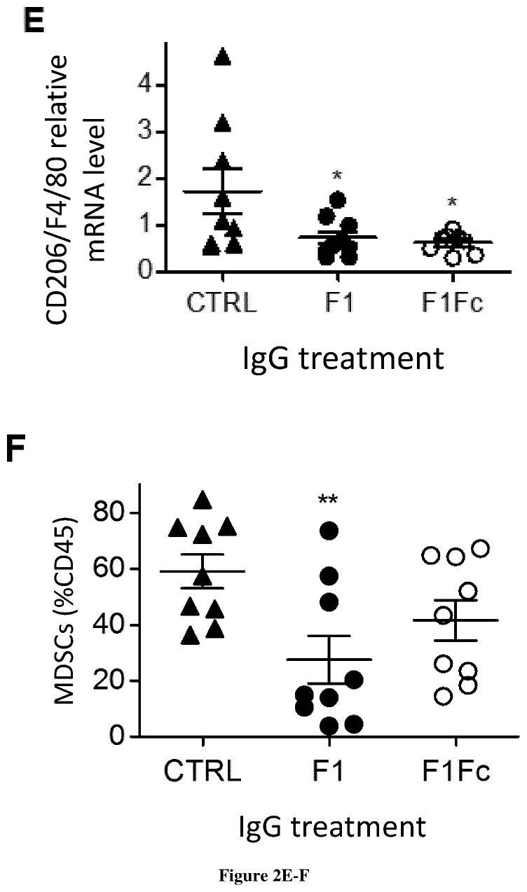

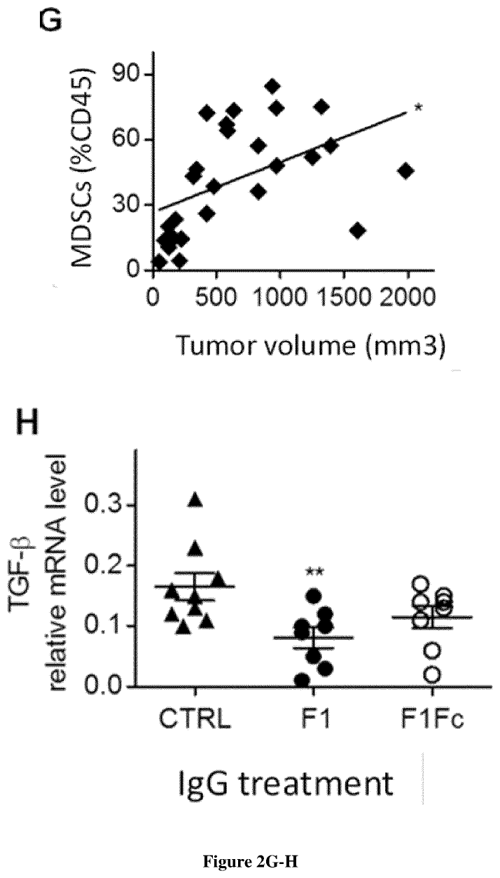

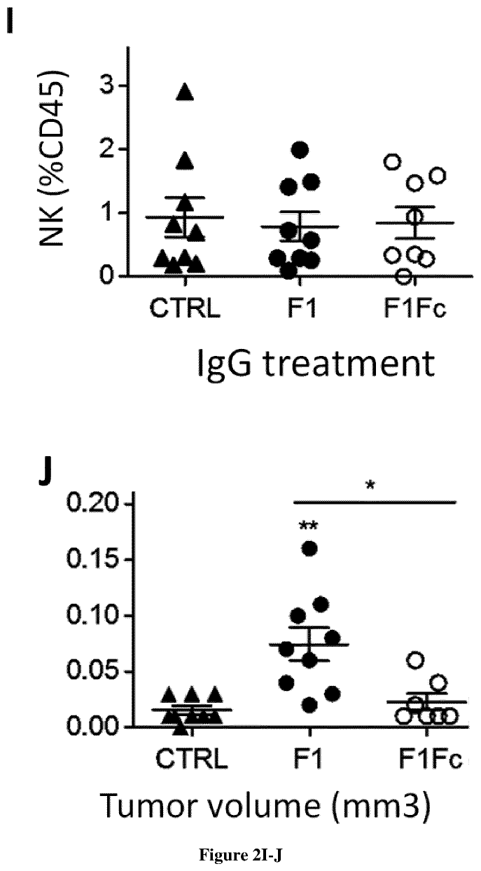

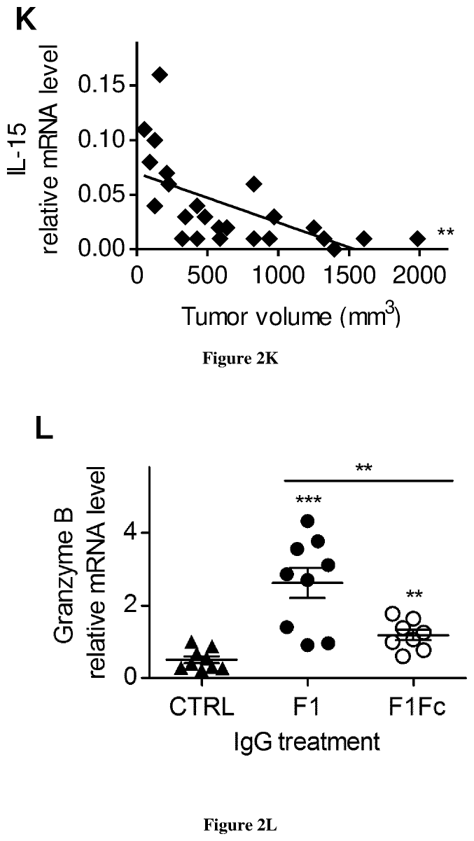

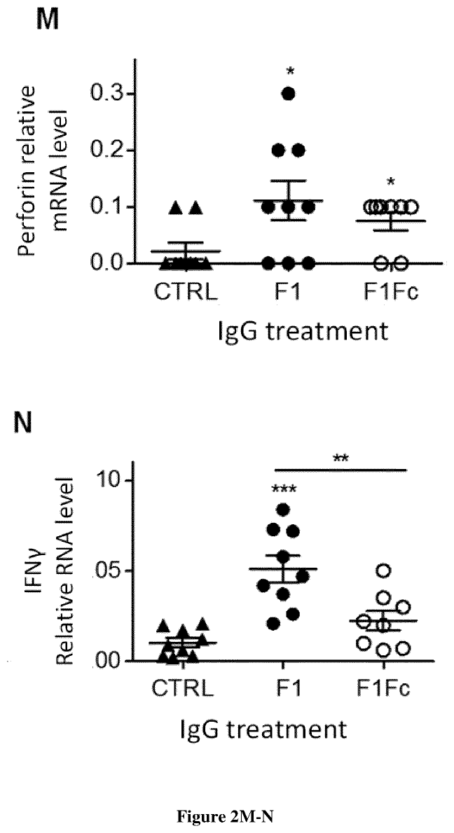

We first confirmed that F1Fc binding to cath-D was comparable to that of F1 (not shown). We then treated mice harboring MDA-MD-231 tumor cell xenografts with F1Fc, F1 or rituximab (CTRL) as before. F1 treatment significantly reduced tumor growth compared with rituximab (FIG. 2A; P<0.001). Conversely, F1Fc effect on tumor growth was reduced compared with F1 (FIG. 2A). At the end of the experiment (day 54), tumor volume was reduced by 63.1% (P=0.01) in the F1 group and only by 32.9% (not significant) in the F1Fc group compared with the rituximab group (FIG. 2B). Thus, the Fc effector functions of F1 are essential for maximal tumor inhibition, corroborating the participation of immune cells in the anti-tumor response induced by anti-cath-D antibody therapy.

We next analyzed tumor immune infiltrates at day 54 by FACS analysis with a specific focus on TAMs and MDSCs, associated with tumor progression and relapse in BC (36, 37). In agreement with the previous IHC results (FIG. 1C), the percentage of F4/80+ CD11b+ macrophages within the immune CD45+ cell population was significantly decreased by 67% in F1-treated animals (P=0.044 compared with the rituximab group) and only by 33% in the F1Fc-treated group (not significant) (FIG. 2C). Moreover, linear regression analysis showed that the percentage of macrophages was significantly correlated with tumor volume in all animals (three treatment groups together) (R2=0.5425, P<0.0001) (FIG. 2D), suggesting that in this model, tumor progression was associated with macrophage enrichment and that the F1 antibody prevented their infiltration. In many tumors including BC, TAMs are M2 polarized, which is associated with pro-tumorigenic functions (37, 39). At day 54, the expression of CD206 mRNA, a M2-associated marker (40), was significantly downregulated by 56.6% (P=0.05) and 62.9% (P=0.04) in MDA-MB-231 tumor xenograft RNA samples from the F1- and F1Fc-treated group, respectively, compared with control (rituximab) (FIG. 2E). This suggests that anti-cath-D antibody monotherapy prevented tumor infiltration by M2 macrophages and that this could have contributed to limit tumor growth.

In addition, the percentage of Gr1+ CD11b+ MDSCs within the immune CD45+ cell population also was significantly decreased by 53.4% (P=0.008) in F1-treated mice and by 29.6% in F1Fc-treated mice (not significant) compared with control (rituximab) (FIG. 2F). The percentage of tumor-infiltrating MDSCs was positively correlated with tumor volume in the whole population (three groups together) (FIG. 2G; P=0.0125). Because of the changes of TAMs and MDSCs, F1 treatment may alter immunosuppressive factors in the tumor microenvironment. Indeed, mRNA expression of the inhibitory cytokine transforming growth factor β (TGFβ) was reduced by 51% in tumors from F1-treated mice (P=0.0099 compared with the rituximab control) and by 30.6% in the F1Fc group (not significant) (FIG. 2H). This strengthened the effect of anti-cath-D antibody therapy on immunosuppressive M2 macrophages and MDSCs. Our data highlight the strong impact of anti-cath-D antibody therapy on the tumor immune microenvironment, leading to a less immunosuppressive microenvironment in MDAMB-231 xenografts.

The Anti-Cath-D Antibody F1 Anti-Tumor Response is Triggered Via NK Cell Activation

NK cells are needed for the efficacy of antibody-based immunotherapies by triggering antibody dependent cell-mediated cytotoxicity (41). To determine the potential implication of NK cells in anticath-D antibody therapy, we quantified by FACS analysis the CD49b+ CD11b+ NK cell population in tumors at the end of treatment (day 54) and found that it was comparable in the F1-, F1Fc- and rituximab treated groups (FIG. 2I). RT-qPCR analysis of the expression of IL-15, a cytokine associated with NK cell activation (42), showed that it was upregulated (up to 209%, P=0.0013 compared with rituximab) in the F1 group, but not in the F1Fc-treated group (P=0.0127 compared with F1) (FIG. 2J). This suggests a causal relationship between the F1 antitumor response and NK cell activation. In agreement, IL-15 mRNA level was inversely correlated with tumor volume in the entire population (three groups together) by linear regression analysis (FIG. 2K; P=0.0013). We then quantified granzyme B (GZMB) and perforin 1 (PRF1) mRNA levels, as a read-out of NK cell activity. GZMB was strongly upregulated (up to 220%) in the F1 group (P=0.0002 compared with rituximab). Although significant, the up-regulation remained modest in the F1Fc group compared with the control group and was significantly reduced compared with the F1-treated group (FIG. 2L; P=0.0076). Similarly, PRFlexpression was increased by 500% in the F1 group compared with control (P=0.033) and slightly less in the F1Fc group (FIG. 2M). Finally, the mRNA expression of the antitumor cytokine IFNγ was upregulated by 494.8% in the F1 group compared with control (FIG. 2N; P<0.0001). This upregulation was significantly reduced in the F1Fc treated group compared with the F1 group (FIG. 2N; P=0.0078). Altogether, our results strongly suggest that the anti-tumor response of the anti-cath-D antibody F1 in MDA-MB-231 xenografts is in part triggered by Fc-dependent mechanisms via NK cell activation through IL15 upregulation, associated with granzyme B and perforin production and the release of IFNγ.

The Anti-Cath-D Antibody F1 Inhibits Growth of Patient-Derived Xenografts of TNBC

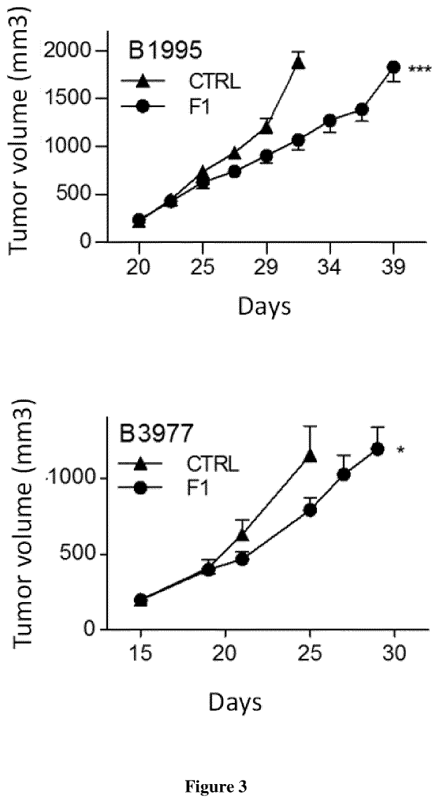

Finally, we tested F1 effect in mice harboring PDXs of TNBC (42). First, quantification by sandwich ELISA in whole cytosolic extracts of five representative TNBC PDXs showed that cath-D concentration varied from 18 to 77 pmol/mg of total protein (data not shown). These values were in the same range as those detected in whole cytosolic extracts prepared from 40 TNBC samples (data not shown). Immunostaining of the B1995 and B3977 primary tumors with an anti-cath-D antibody confirmed that cath-D was detected in tumor cells and microenvironment (data not shown), as previously observed with the TNBC TMA (data not shown). These results indicate these PDX models are representative of the disease, at least concerning cath-D expression. We then engrafted athymic nude mice with PDX B1995 or PDX B3977, the two PDXs showing the fastest growth in nude mice (average passage duration for the first three passages: 46 days for B1995 and 42 days for B3977) (data not shown). Tumor volume increase was significantly slowed down in mice treated with F1 compared with control (FIG. 3).

In conclusion, inventors have demonstrated for the first time that cath-D inhibits the tumor recruitment of immunosuppressive tumor-associated macrophages M2 and myeloid-derived suppressor cells. Furthermore, their preclinical proof-of-concept study validates the feasibility and efficacy of an immunomodulatory antibody-based strategy against cath-D to treat patients with TNBC.

### REFERENCES

Throughout this application, various references describe the state of the art to which this invention pertains. The disclosures of these references are hereby incorporated by reference into the present disclosure.
1. Bianchini G, Balko J M, Mayer I A, Sanders M E, Gianni L. Triple-negative breast cancer: challenges and opportunities of a heterogeneous disease. Nat Rev Clin Oncol. 2016; 13(11):674-90.
2. Schmid P, Adams S, Rugo H S, Schneeweiss A, Barrios C H, Iwata H, et al. Atezolizumab and Nab-Paclitaxel in Advanced Triple-Negative Breast Cancer. The New England journal of medicine. 2018.
3. Vignon F, Capony F, Chambon M, Freiss G, Garcia M, Rochefort H. Autocrine growth stimulation of the MCF 7 breast cancer cells by the estrogen-regulated 52 K protein. Endocrinology. 1986; 118(4):1537-45.
4. Foekens J A, Look M P, Bolt-de Vries J, Meijer-van Gelder M E, van Putten W L, Klijn J G. Cathepsin-D in primary breast cancer: prognostic evaluation involving 2810 patients. Br J Cancer. 1999; 79(2):300-7.
5. Ferrandina G, Scambia G, Bardelli F, Benedetti Panici P, Mancuso S, Messori A. Relationship between cathepsin-D content and disease-free survival in node-negative breast cancer patients: a meta-analysis. Br J Cancer. 1997; 76(5):661-6.
6. Glondu M, Liaudet-Coopman E, Derocq D, Platet N, Rochefort H, Garcia M. Down-regulation of cathepsin-D expression by antisense gene transfer inhibits tumor growth and experimental lung metastasis of human breast cancer cells. Oncogene. 2002; 21(33):5127-34.
7. Vetvicka V, Benes P, Fusek M. Procathepsin D in breast cancer: what do we know? Effects of ribozymes and other inhibitors. Cancer Gene Ther. 2002; 9(10):854-63.
8. Laurent-Matha V, Maruani-Herrmann S, Prebois C, Beaujouin M, Glondu M, Noel A, et al. Catalytically inactive human cathepsin D triggers fibroblast invasive growth. J Cell Biol. 2005; 168(3):489-99.
9. Beaujouin M, Prebois C, Derocq D, Laurent-Matha V, Masson O, Pattingre S, et al. Pro-cathepsin D interacts with the extracellular domain of the beta chain of LRP1 and promotes LRP1-dependent fibroblast outgrowth. J Cell Sci. 2010; 123(Pt 19):3336-46.
10. Berchem G, Glondu M, Gleizes M, Brouillet J P, Vignon F, Garcia M, et al. Cathepsin-D affects multiple tumor progression steps in vivo: proliferation, angiogenesis and apoptosis. Oncogene. 2002; 21(38):5951-5.
11. Pranjol M Z I, Gutowski N J, Hannemann M, Whatmore J L. Cathepsin D non-proteolytically induces proliferation and migration in human omental microvascular endothelial cells via activation of the ERK1/2 and PI3K/AKT pathways. Biochimica et biophysica acta. 2017; 1865(1):25-33.
12. Hasilik A, von Figura K, Conzelmann E, Nehrkorn H, Sandhoff K. Lysosomal enzyme precursors in human fibroblasts. Activation of cathepsin D precursor in vitro and activity of beta-hexosaminidase A precursor towards ganglioside GM2. Eur J Biochem. 1982; 125(2):317-21.
13. Benes P, Vetvicka V, Fusek M. Cathepsin D—many functions of one aspartic protease. Critical reviews in oncology/hematology. 2008; 68(1):12-28.
14. Masson O, Bach A S, Derocq D, Prebois C, Laurent-Matha V, Pattingre S, et al. Pathophysiological functions of cathepsin D: Targeting its catalytic activity versus its protein binding activity? Biochimie. 2010; 92(11):1635-43.
15. Khalkhali-Ellis Z, Hendrix M J. Two Faces of Cathepsin D: Physiological Guardian Angel and Pathological Demon. Biology and medicine. 2014; 6(2).
16. Wolf M, Clark-Lewis I, Buri C, Langen H, Lis M, Mazzucchelli L. Cathepsin D specifically cleaves the chemokines macrophage inflammatory protein-1 alpha, macrophage inflammatory protein-1 beta, and SLC that are expressed in human breast cancer. The American journal of pathology. 2003; 162(4):1183-90.
17. Hasan L, Mazzucchelli L, Liebi M, Lis M, Hunger R E, Tester A, et al. Function of liver activation-regulated chemokine/CC chemokine ligand 20 is differently affected by cathepsin B and cathepsin D processing. Journal of immunology. 2006; 176(11):6512-22.
18. Laurent-Matha V, Huesgen P F, Masson O, Derocq D, Prebois C, Gary-Bobo M, et al. Proteolysis of cystatin C by cathepsin D in the breast cancer microenvironment. FASEB J. 2012.
19. Maynadier M, Farnoud R, Lamy P J, Laurent-Matha V, Garcia M, Rochefort H. Cathepsin D stimulates the activities of secreted plasminogen activators in the breast cancer acidic environment. Int J Oncol. 2013; 43(5):1683-90.
20. Vetvicka V, Vetvickova J, Hilgert I, Voburka Z, Fusek M. Analysis of the interaction of procathepsin D activation peptide with breast cancer cells. Int J Cancer. 1997; 73(3):403-9.
21. Derocq D, Prebois C, Beaujouin M, Laurent-Matha V, Pattingre S, Smith G K, et al. Cathepsin D is partly endocytosed by the LRP1 receptor and inhibits LRP1-regulated intramembrane proteolysis. Oncogene. 2012; 31(26):3202-12.
22. Siintola E, Partanen S, Stromme P, Haapanen A, Haltia M, Maehlen J, et al. Cathepsin D deficiency underlies congenital human neuronal ceroid-lipofuscinosis. Brain. 2006; 129(Pt 6):1438-45.
23. Anantaraju H S, Battu M B, Viswanadha S, Sriram D, Yogeeswari P. Cathepsin D inhibitors as potential therapeutics for breast cancer treatment: Molecular docking and bioevaluation against triple-negative and triple-positive breast cancers. Molecular diversity. 2016; 20(2):521-35.
24. Freiss G, Vignon F, Rochefort H. Characterization and properties of two monoclonal antibodies specific for the Mr 52,000 precursor of cathepsin D in human breast cancer cells. Cancer Res. 1988; 48(13):3709-15.
25. Saadoun H, Lamy P J, Thezenas S, Pouderoux S, Bibeau F, Montels F, et al. Prognostic impact of the inclusion of uPA/PAI-1 tumor levels in the current adjuvant treatment decision-making for early breast cancer. Future Oncol. 2014; 10(2):195-209.
26. du Manoir S, Orsetti B, Bras-Goncalves R, Nguyen T T, Lasorsa L, Boissiere F, et al. Breast tumor PDXs are genetically plastic and correspond to a subset of aggressive cancers prone to relapse. Molecular oncology. 2014; 8(2):431-43.
27. Webb B, Sali A. Comparative Protein Structure Modeling Using MODELLER. Current protocols in bioinformatics. 2014; 47:5 6 1-32.
28. Bourquard T, Landomiel F, Reiter E, Crepieux P, Ritchie D W, Aze J, et al. Unraveling the molecular architecture of a G protein-coupled receptor/beta-arrestin/Erk module complex. Scientific reports. 2015; 5:10760.
29. Gyorffy B, Lanczky A, Eklund A C, Denkert C, Budczies J, Li Q, et al. An online survival analysis tool to rapidly assess the effect of 22,277 genes on breast cancer prognosis using microarray data of 1,809 patients. Breast cancer research and treatment. 2010; 123(3):725-31.
30. Turtoi A, De Pauw E, Castronovo V. Innovative proteomics for the discovery of systemically accessible cancer biomarkers suitable for imaging and targeted therapies. The American journal of pathology. 2011; 178(1):12-8.
31. Philibert P, Stoessel A, Wang W, Sibler A P, Bec N, Larroque C, et al. A focused antibody library for selecting scFvs expressed at high levels in the cytoplasm. BMC Biotechnol. 2007; 7:81.
32. Robin G, Martineau P. Synthetic customized scFv libraries. Methods Mol Biol. 2012; 907:109-22.
33. Baldwin E T, Bhat T N, Gulnik S, Hosur M V, Sowder R C, 2nd, Cachau R E, et al. Crystal structures of native and inhibited forms of human cathepsin D: implications for lysosomal targeting and drug design. Proc Natl Acad Sci USA. 1993; 90(14):6796-800.
34. Weiner G J. Building better monoclonal antibody-based therapeutics. Nature reviews Cancer. 2015; 15(6):361-70.
35. Nimmerjahn F, Ravetch J V. Translating basic mechanisms of IgG effector activity into next generation cancer therapies. Cancer Immun. 2012; 12:13.
36. Hristodorov D, Fischer R, Joerissen H, Muller-Tiemann B, Apeler H, Linden L. Generation and comparative characterization of glycosylated and aglycosylated human IgG1 antibodies. Molecular biotechnology. 2013; 53(3):326-35.
37. Zhao X, Qu J, Sun Y, Wang J, Liu X, Wang F, et al. Prognostic significance of tumor-associated macrophages in breast cancer: a meta-analysis of the literature. Oncotarget. 2017; 8(18):30576-86.
38. Safarzadeh E, Orangi M, Mohammadi H, Babaie F, Baradaran B. Myeloid-derived suppressor cells: Important contributors to tumor progression and metastasis. Journal of cellular physiology. 2018; 233(4):3024-36.
39. Tariq M, Zhang J, Liang G, Ding L, He Q, Yang B. Macrophage Polarization: Anti-Cancer Strategies to Target Tumor-Associated Macrophage in Breast Cancer. Journal of cellular biochemistry. 2017; 118(9):2484-501.
40. Scodeller P, Simon-Gracia L, Kopanchuk S, Tobi A, Kilk K, Saalik P, et al. Precision Targeting of Tumor Macrophages with a CD206 Binding Peptide. Scientific reports. 2017; 7(1):14655.
41. Souza-Fonseca-Guimaraes F. NK cell-based immunotherapies: awakening the innate anti-cancer response. Discovery medicine. 2016; 21(115):197-203.
42. Steel J C, Waldmann T A, Morris J C. Interleukin-15 biology and its therapeutic implications in cancer. Trends in pharmacological sciences. 2012; 33(1):35-41.
43. Huang L, Liu Z, Chen S, Liu Y, Shao Z. A prognostic model for triple-negative breast cancer patients based on node status, cathepsin-D and Ki-67 index. PloS one. 2013; 8(12):e83081.
44. Vetvicka V, Vetvickova J, Fusek M. Anti-human procathepsin D activation peptide antibodies inhibit breast cancer development. Breast cancer research and treatment. 1999; 57(3):261-9.
45. Nimmerjahn F, Gordan S, Lux A. FcgammaR dependent mechanisms of cytotoxic, agonistic, and neutralizing antibody activities. Trends in immunology. 2015; 36(6):325-36.
46. Fuxe J, Karlsson M C. TGF-beta-induced epithelial-mesenchymal transition: a link between cancer and inflammation. Seminars in cancer biology. 2012; 22(5-6):455-61.
47. Georgoudaki A M, Prokopec K E, Boura V F, Hellqvist E, Sohn S, Ostling J, et al. Reprogramming Tumor-Associated Macrophages by Antibody Targeting Inhibits Cancer Progression and Metastasis. Cell reports. 2016; 15(9):2000-11.
48. Gabrilovich D I, Nagaraj S. Myeloid-derived suppressor cells as regulators of the immune system. Nature reviews Immunology. 2009; 9(3):162-74.
49. Ito R, Takahashi T, Katano I, Ito M. Current advances in humanized mouse models. Cellular & molecular immunology. 2012; 9(3):208-14.

1. A method of treating a hyperproliferative disease in a subject in need thereof, comprising
administering to the subject a therapeutically effective amount of a human anti-cathepsin-D antibody which inhibits the tumor recruitment of immunosuppressive tumor-associated macrophages M2 and myeloid-derived suppressor cells.

2. The method according to claim 1 wherein said antibody comprises: a) a heavy chain CDR1 comprising the amino acid sequence of SEQ ID NO: 2, b) a heavy chain CDR2 comprising the amino acid sequence of SEQ ID NO: 3, c) a heavy chain CDR3 comprising the amino acid sequence of SEQ ID NO: 4; d) a light chain CDR1 comprising the amino acid sequence of SEQ ID NO: 6; e) a light chain CDR2 comprising the amino acid sequence of SEQ ID NO: 7; and f) a light chain CDR3 comprising the amino acid sequence of SEQ ID NO: 8.

3. The method according to claim 1 wherein said antibody comprises: a) a heavy chain CDR1 comprising the amino acid sequence of SEQ ID NO: 10, b) a heavy chain CDR2 comprising the amino acid sequence of SEQ ID NO: 11, c) a heavy chain CDR3 comprising the amino acid sequence of SEQ ID NO: 12; d) a light chain CDR1 comprising the amino acid sequence of SEQ ID NO: 14; e) a light chain CDR2 comprising the amino acid sequence of SEQ ID NO: 15; and f) a light chain CDR3 comprising the amino acid sequence of SEQ ID NO: 16.

4. A nucleic acid sequence encoding a heavy chain or light chain of a human anti-cathepsin-D antibody which inhibits the tumor recruitment of immunosuppressive tumor-associated macrophages M2 and myeloid-derived suppressor cells.

5. A vector comprising a nucleic acid sequence according to claim 4.

6. A host cell comprising a nucleic acid sequence according to claim 4 or a vector comprising the nucleic acid sequence.

7. (canceled)

8. The method according to claim 1, wherein the hyperproliferative disease is cancer.

9. The method of claim 8 wherein the cancer is triple-negative breast cancer (TNBC).

10. A pharmaceutical composition comprising a human anti-cathepsin-D antibody which inhibits the tumor recruitment of immunosuppressive tumor-associated macrophages M2 and myeloid-derived suppressor cells.

11. (canceled)

12. The method according to claim 1, further comprising administering to the subject an immune checkpoint inhibitor.

13. (canceled)

14. A method for treating cancer in a subject in need thereof comprising administering to said subject a therapeutically effective amount of a human anti-cathepsin-D antibody or a fragment thereof.


SEQUENCE LISTING (16 sequences)

SEQ ID NO: 1 - protein, 117 aa, Artificial
  Synthetic VH-F1
EVQLVESGGSLVKPGGSLRLSCAASGFTSNNYMNWVRQAPGKGLEWISYISGSSRYISYADFVKGRFTISRDNATNSLYLQMNSLRAEDTAVYYCVRSSNSGGMDVWGRGTLVTVSS

SEQ ID NO: 2 - protein, 8 aa, Artificial
  Synthetic VH-CDR1-F1
GFTFSNNY

SEQ ID NO: 3 - protein, 8 aa, Artificial
  Synthetic VH-CDR2-F1
ISGSSRYI

SEQ ID NO: 4 - protein, 11 aa, Artificial
  Synthetic VH-CDR3-F1
VRSSNSGGMDV

SEQ ID NO: 5 - protein, 110 aa, Artificial
  Synthetic VL-F1
QSVLTQPASVSGSPGQSITISCAGTSSDVGGYYGVSWYQQHPGKAPKLMIYYDSYRPSGVSNRFSGSKSGNTASLTISGLQAEDEADYYCSSYTSNSTRVFGGGTKLAVL

SEQ ID NO: 6 - protein, 9 aa, Artificial
  Synthetic VL-CDR1-F1
SSDVGGYYG

SEQ ID NO: 7 - protein, 3 aa, Artificial
  Synthetic VL-CDR2-F1
GDS

SEQ ID NO: 8 - protein, 10 aa, Artificial
  Synthetic VL-CDR3-F1
SSYTSNSTRV

SEQ ID NO: 9 - protein, 120 aa, Artificial
  Synthetic VH-E2
EVQLVESGGSLVKPGGSLRLSCAASGFTFSNSYMNWVRQAPGKGLEWISYISGSSRYSYADFVKGRFTISRDNATNSLYLQMNSLRAEDTAVYYCVRSSNSYFGGGMDVWGRGTLVTVSS

SEQ ID NO: 10 - protein, 8 aa, Artificial
  Synthetic VH-CDR1-E2
GFTFSNSY

SEQ ID NO: 11 - protein, 8 aa, Artificial
  Synthetic VH-CDR2-E2
ISGSSRYI

SEQ ID NO: 12 - protein, 14 aa, Artificial
  Synthetic VH-CDR3-E2
VRSSNSYFGGGMDV

SEQ ID NO: 13 - protein, 110 aa, Artificial
  Synthetic VL-E2
QSVLTQPASVSGSPGQSITISCAGTSSDVGGSYGVSWYQQHPGKAPKLMIYGDSYRPSGVSNRFSGSKSGNTASLTISGLQAEDEADYYCSSYTNYSTRVFGGGTKLAVL

SEQ ID NO: 14 - protein, 9 aa, Artificial
  Synthetic VL-CDR1-E2
SSDVGGSYG

SEQ ID NO: 15 - protein, 3 aa, Artificial
  Synthetic VL-CDR2-E2
GDS

SEQ ID NO: 16 - protein, 10 aa, Artificial
  Synthetic VL-CDR3-E2
SSYTNYSTRV